(19) United States
(12) Patent Application Publication
(10) Pub. No.: US 2025/0194606 A1
(43) Pub. Date: Jun. 19, 2025

(54) A METHOD FOR REDUCING THE SUPPRESSION OF CORN GROWTH DUE TO WATERLOGGING STRESS

(71) Applicant: CH Biotech R&D Co., Ltd., Nantou City (TW)

(72) Inventors: Kai XIA, Nantou (TW); Ya-Wen HUANG, Taichung (TW); Cyun-Jhe YAN, Taoyuan (TW)

(73) Assignee: CH Biotech R&D Co., Ltd., Nantou City (TW)

(21) Appl. No.: 18/658,824

(22) Filed: May 8, 2024

(30) Foreign Application Priority Data
Dec. 15, 2023 (TW) 112149129

(51) Int. Cl. A01N 63/10; A01H 3/00; A01N 37/46

(52) U.S. Cl. CPC A01N 63/10; A01H 3/00; A01N 37/46

(57) ABSTRACT

Present invention teaches the method of using a keratin hydrolysis peptide (“KHP”) solution to reduce the suppression of corn growth due to waterlogging stress. By selectively choosing specific weights of feathers and water, and treating the mixture to a high-temperature high-pressure hydrolysis process, the resulting solution is confirmed to contain at least 253 peptides and then infused to the soil containing the corn seeds or seedlings; the solution can also be sprayed to the leaf surface of corn seedlings at a growth period of the corn seedlings. Optionally, the KHP solution can be diluted by water, as disclosed in the specification, for applying to the soil or for spraying to leaf surface of corn seedlings.

### PRIORITY CLAIM TO FOREIGN APPLICATION

Applicant hereby makes priority claim to a Taiwan application, number 112149129, having the Taiwan filing date of Dec. 15, 2023.

### BACKGROUND OF THE INVENTION

Present invention disclosed and claimed the method and application of a keratin hydrolysis peptide (“KHP”) solution to reduce the suppression of corn growth due to flood or waterlogging stress.

Hydrolyzed keratin has long been used to strengthen hairs, reduce hair splitting and breakage. Other beneficial uses include skin moisturization and wound healing. Keratin hydrolysate has also been known to function as a biofertilizer, boosting plants' growth by enhancing the plants' ability to receive and utilize nutrients, including commonly applied fertilizers.

The KHP solution is made by a hydrolysis process using feathers and water, via a high-temperature and high-pressure process, resulting in a solution that has many beneficial applications in the fields of horticulture, agriculture and potentially other farming businesses.

Among the embodiments disclosed and claimed in this application, one specific embodiment used feathers only, without water, to be treated with the high-temperature and high-pressure process as taught herein to produce a specific version of the KHP

### Solution

Farm land floods in recent years are one of the adverse conditions that cause global economic losses to crops, including corns. The climate change of late brought about many instances of excessive rains and floods that are especially bad of farmers to effectively grow their corn crops when the corn plants are damaged due to waterlogging stress, which leads to lack of soil oxygen and the accumulation of certain toxic content in the soil including Mn²⁺ and Fe²⁺, further causing the metabolism of the plants to fail and cell structure decay, and, worst of all, the death of the whole plants.

During the time of seeding and germination, if the corn plants are flooded, the growth will be stunted, leading to many developmental defects, including the limited photosynthesis activities, lower chlorophyll counts, and insufficient nutrient intake and production of generally healthy biomass, both above-ground and under-ground.

The growth point of corn seedlings is located below soil surface, and is very sensitive to flooding. During the time between seeding and sprouting, the cord seedlings are most sensitive to flooding; if the corn seedlings are flooded for more than 48 hours during the sensitive period, the growth point will suffer irreparable damages due to lack of oxygen; the root system will not be able to develop properly, and will not able to transport nutrients upwards toward the stems and above ground portion. Ultimately, the whole corn plant will start yellowing, and then, without nutrient to support further growth, die.

Many researches have been done to find solutions that can alleviate the damages caused by such waterlogging stress to corn. The inventors of present invention discovered that by applying specific KHP solutions, in the method steps as disclosed herein, such stress is greatly reduced and the damages remedied, as proven by the experiments done.

### SUMMARY OF THE INVENTION

The keratin solution is primarily based upon feather, which contains 85-91% keratin, 13-15% organic nitrogen, 1.6-2% organic sulfur, as well as other materials. The high keratin content has drawn many prior researches that work to break down, by enzyme, chemical agents, or fermentation process, into peptides, amino acids and other smaller molecules that can be used for animal feeds, plant fertilizers, and cultivation bases.

Around 2019, Nurdiawati, et al, came up with a hydrolysis process, by the mixture of a-amylase and protease to hydrolyze feather waste, resulting in a mixture of amino acids, fatty acids, and sugars. Nurdiawati experimented and adopted certain specific high-temperature and high-pressure setting in the hydrolysis process and discovered that the resulting solution, when mixed with some potassium and other minerals, can boost the growth of Pogostemon cablin and Vigna radiata, as reported in International Journal of Recycling or Organic Waste in Agriculture (8:221-232, 2019).

The inventors of present application, under the aegis of CH Biotech, developed and selected different feather and water compositions to perform the hydrolysis at higher temperature and higher-pressure setting, resulting with different keratin hydrolysis peptide (“KHP”) solution that can be used on different crops/plants.

The selected embodiment of present invention uses a mixture of water and feathers, and subject the mixture to a thermal hydrolysis process to create KHP solutions based upon temperature/pressure parameters as noted below.

The inventors used the Dionex UltiMate 3000 UPLC to separate the peptides; an analysis is done via Thermo Orbitrap Fusion Lumos Tribrid Orbitrap mass spectrometry to identify the peptides, which are then subsequently confirmed by looking up the BIOPEP-UWM database.

The solutions can be infused to the soil containing the corn seeds before seeding; alternatively, the solutions can be infused to the soil during the time of seeding the corn.

Another way of applying the KHP solutions is by spraying to the leaf surface of young corn plants during the seedling stage.

The solution can be diluted by water, at 50 to 500 ratio by volume, and then infuse to the soil or spray to the leaf surface.

### BRIEF DESCRIPTION OF DRAWINGS

The accompanying drawings, figures and tables, which are incorporated in and constitute a part of this specification, illustrate and exemplify the preferred embodiments of the invention. Together with the description, serve to explain the principles of the invention.

Table I (in Sequence Listing XML format) shows the at least 253 peptides and its annotated sequences for the solution generated in accordance with the disclosure of this application. The Sequence Listing XML file complies with the WIPO ST.26 requirements.

Applicant hereby incorporated by reference said Sequence Listing XML file in its entirety as part of the disclosure and specification of the present application.

The Sequence Listing XML file is identified as follows:
File name: Table-I-253_sequence
Created on: Mar. 17, 2024
Size: 216 KB

FIG. 1A compares the images of root development in different groups. T2 root growth was inhibited due to flood; the roots in T3 and T4 show better root development induced by application of KHP solution.

FIG. 1B compares the root dry weights in different groups; the roots in T3 and T4, boosted by the application of the KHP solution, show vastly more weights than the T2 group that's suffered from the waterlogging stress.

FIG. 2A presents the photo images as comparisons of the growth statures of the corns in different groups.

FIG. 2B shows the comparison of the plant heights of the different groups.

FIG. 2C shows the comparison of the stem widths of the different groups.

FIG. 2D shows the comparison of the fresh weights for the above-ground biomass.

FIG. 2E shows the comparison of the dry weights for the above-ground biomass.

FIG. 3A shows the comparison of leaf areas measured from the 3^{rd}, 4^{th} and 5^{th} leaves.

FIG. 3B shows the comparison of SPAD readings obtained from the 3^{rd}, 4^{th} and 5^{th} leaves.

FIG. 4A shows the comparison of dry weights of the above-ground biomass, with KHP applications done by both soil infusion and leaf spray to reduce the waterlogging suppression.

FIG. 4B shows the comparison of plant heights, with KHP applications done by soil infusion (T9 group) and leaf spray (T7 and T8 groups).

FIG. 4C shows the comparison of stem widths, with KHP applications done by soil infusion (T9 group) and leaf spray (T7 and T8 groups).

FIG. 5A shows the comparison of total leaf areas among all the groups.

FIG. 5B shows the comparison of SPAD readings obtained from the 5^{th} leaves.

FIG. 5C shows the comparison of SPAD readings obtained from the 6^{th} leaves.

### DETAILED DESCRIPTION OF THE INVENTION

The keratin hydrolysis peptide (“KHP”) solution of present invention is made by a high-temperature and high-pressure process to treat a mixture of water and feathers as shown in the parameters herein.

The mixture ratio, temperature, pressure and duration parameters are shown herein:

| | | | Water | | | | | |
|---|---|---|---|---|---|---|---|---|
| | | | content | | | | | |
| | Feather | Water | in feather | Pressure | Temp. | Time | Mass | Concen. |
| | (kg) | (kg) | (%) | (kg/cm²) | (° C.) | (min) | (Da) | (ppm) |
| | 66 | 44 | 50% | 16 | 195 | 40 | 593.3~3828.0 | 200000 |
| KHP-A | 50 | 40 | 50% | 12 | 185 | 80 | 593.3~3508.9 | 301500 |
| KHP-B | 70 | 0 | 46% | 13 | 180 | 40 | 705.9~3194.7 | 381250 |

The keratin hydrolysis peptide (“KHP”) solution in the chosen embodiment, noted as KHP-A, takes the steps of:
a. Preparing the KHP solution by mixing 50 kg of feathers whose content is 50% water with 40 kg of water in a sealed container;
b. hydrolyzing the mixture in the container with a temperature and pressure setting of 185° C. and 12 kg/cm² for a duration of 80 minutes;
c. using a mass spectrometer to confirm the combination of peptides in the solution to contain at least 253 peptides as listed in the specification where their molecular masses are between 500 and 4,000 Daltons.

The KHP-A solution of the first embodiment is further filtered and concentrated to 301,500 ppm concentration.

A second embodiment of keratin hydrolysis peptide (KHP) solution, noted as KHP-B, is made by 70 kg of feathers, with the feathers' water content being 46%, without adding any water, treated by the steps of:
a. stirring the feathers in a sealed container;
b. hydrolyzing the feathers in the container with a temperature and pressure setting of 180° C. and 13 kg/cm² for a duration of 40 minutes;
c. using a mass spectrometer to confirm the combination of peptides in the solution to contain at least 253 peptides as listed in the specification whereby their molecular masses are between 705.9 and 3,194.7 Dalton.

The KHP-B solution of the second embodiment is further filtered and concentrated to 381,250 ppm concentration.

The confirmation of some of the 253 peptides is further done by referencing the BIOPEP-UWM database.

The KHP solution derived can be diluted with water by volume at the ratio of 1:50-500. Herein the applicant will use the convention of 50× to denote dilution by 50 times of water; 500× would similarly denote 500 times of water.

The inventors chose the corn species of YuMeiJen to test the method of KHP application disclosed herein, in a greenhouse environment, with day and night temperatures set at 25° C./23° C.

To conduct the effectiveness of soil infusion of the KHP solution when the seeding is done, four (4) groups are defined: T1 is normal environment group where the corns will go through unflooded setting, aka normal, setting. There are three (3) groups of corn plantings that are flooded: T2, T3 and T4.

All four groups are given common fertilizer, TaiFei Instant 43 (N-P-K 15-15-15).

The 3 flood groups' soil infusions are different. T3 is given 50 ml of KHP-A 100× per pot; T4 is given 50 ml KHP-A 50× per pot. T2 group is the check group; instead of giving KHP solution, only the same 50 ml/pot of water is given to the soil containing corn seeds.

For the 3 groups T2, T3 and T4, the pots are flooded for 11 days from seeding to V2 stage of the seedling (second leaf circle is visible), then the corns are return to normal growth setting.

The grouping conditions are summarized below:

| | Group | Dosage (/pot) | Setting |
|---|---|---|---|
| T1 | Normal Environment | Water 50 ml | Normal growth |
| T2 | Check | Water 50 ml | 11 days of flood |
| T3 | KHP-A 100x | KHP 50 ml | from seeding to V2 |
| T4 | KHP-A 50x | KHP 50 ml | stage |

On the seventh day after the flooded groups are returned to normal growth conditions, the inventors gather and measure the relevant indicators and take photos and noted the results as further stated herein.

To measure weights, both dry and fresh weights, the inventors use digital scale (AP224x Shimadazu) to get the precise reading. To obtain the SPAD (Soil-Plant Analysis Development) readings, the inventors relied upon the instrument of SPAD 502 Plus. The inventors utilized WinFOLIA Pro LA 2400 (Regent) to obtain and compute leaf surface areas.

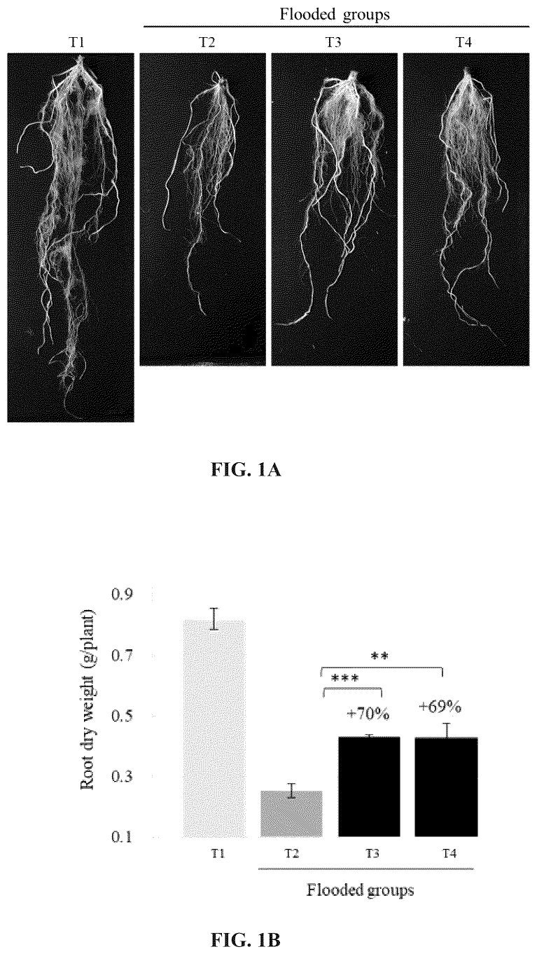

From the photos taken, the root systems' development are noted in FIG. 1A. As can be seen, the T3 and T4 groups' root development is better than that of T2 where the corns were flooded without any remedial assistance from the KHP solutions. T1 shows the healthier development of root system in an unflooded, normal, setting.

The root's dry weights were gathered and compared and tabulated into FIG. 1B. As can be seen, both T3 and T4 groups' dry weights are roughly 70% more than that of the T2 group.

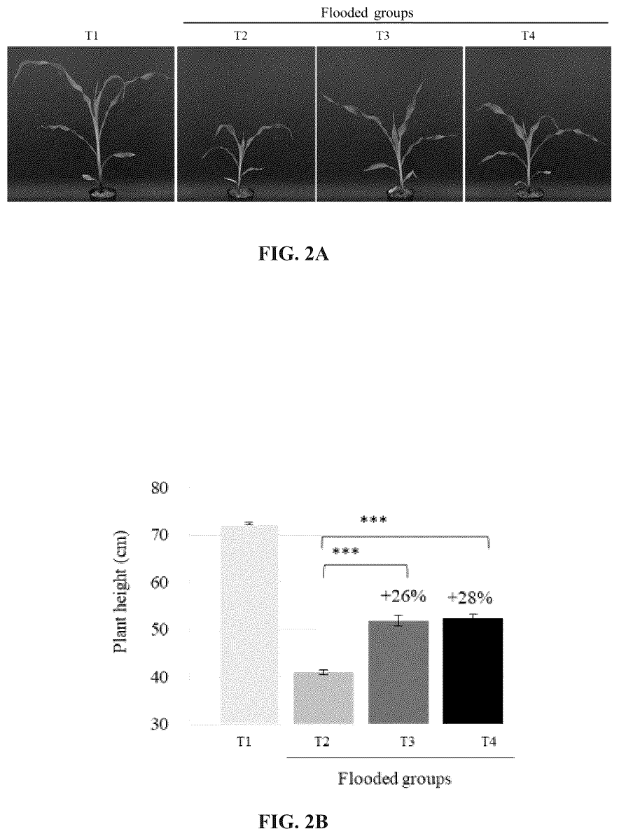

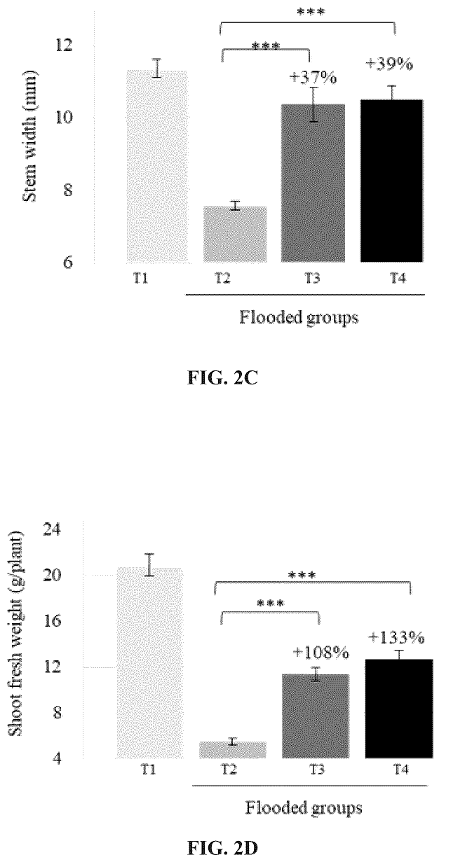

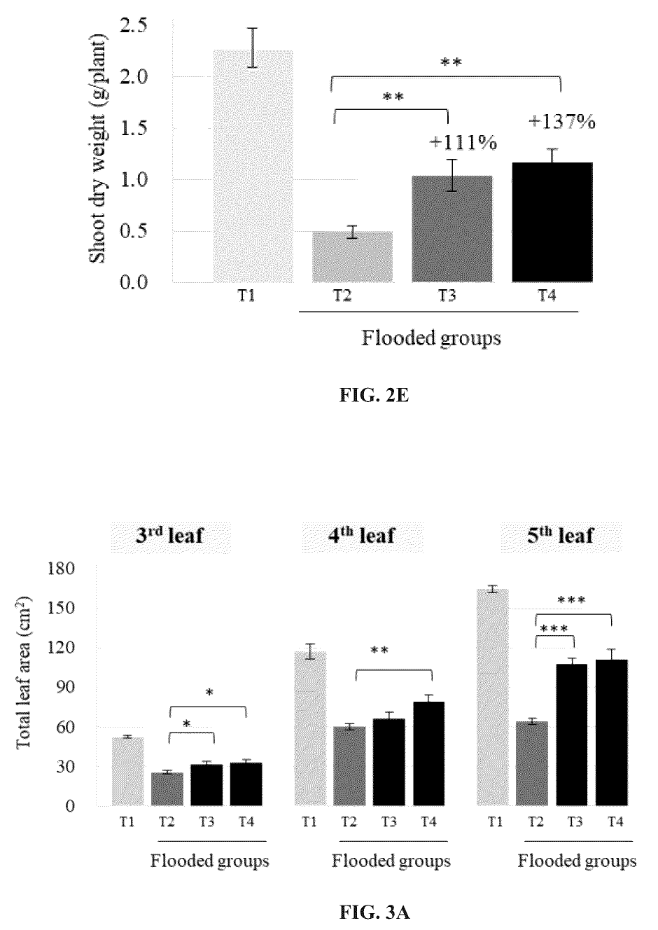

The above-ground growth statures are photographed and noted in FIG. 2A. It can be seen that T3 and T4 groups have stronger and healthier stature than that of T2.

The plant heights of the 4 groups are noted in FIG. 2B. T3's height is 26% better than T2; T4's height is 28% better than T2.

For the stem widths, the results are noted in FIG. 2C. As can be seen, T3's stem width is 32% better than that of T2; T4's stem width is better than that of T2.

For the above-ground biomass fresh weights, the results are noted in FIG. 2D. As can be seen, T3's above-ground fresh weight is 108% better than that of T2; T4's above-ground fresh weight is 133% better than T2.

The inventors further compare the dry weights of the above-ground biomass and noted the results in FIG. 2E. As shown, T3's above-ground biomass dry weight has 111% increase over that of T2; T4 has 137% increase over that of T2.

The inventors also checked the effectiveness of KHP application to leaf development by measuring the SPAD reading and leaf areas of the 3^{rd}, 4^{th} and 5^{th} leaves of the corn seedlings.

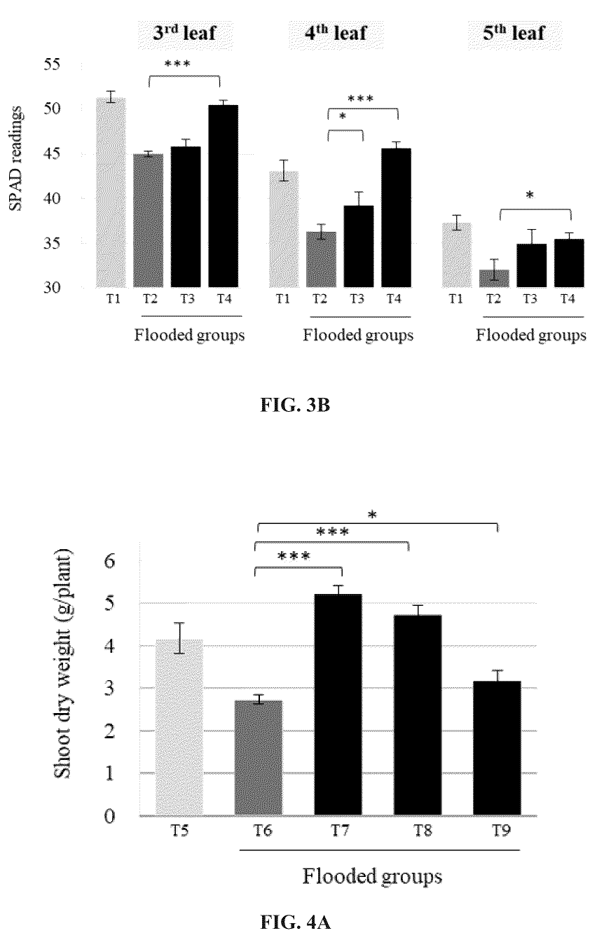

FIG. 3A shows the leaf area comparisons done on the 3^{rd}, 4^{th} and 5^{th} leaves. The effect of reducing the waterlogging stress is made very obvious from the 5^{th} leaf bar chart where the T3 and T4 leaf areas show substantial increase over that of T2.

For the SPAD analysis, the readings are tabulated into FIG. 3B. As can been seen, the application of KHP solution remedied the injuries caused by the flooding. For the SPAD reading in the 3^{rd} leaf bar chart, the T4 group's SPAD is very close to the normal group's reading, tantamount to not suffering flood.

To find out the effectiveness of applying KHP solution by spraying to the leaf surface to the flooded corn plants, the inventors defined another five groups: T5 through T9 where T5 is the normal growth setting, with T6, T7, T8 and T8 are the flooded groups, where T6 is not given KHP solutions.

T7 group, at the seedling V2 stage, is treated with leaf spraying 0.8 ml of KHP-A 500× solution. T8 group, at the seedling V2 stage, is treated with leaf spraying of 0.8 ml of KHP-B 500× solution. T9 group, at the seedling V2 stage, is infused with 50 ml of KHP-A 100× solution to the soil.

The conditions of the T5-T9 are summarized herein:

| | Group | Dosage (/pot) | Setting |
|---|---|---|---|
| T5 | Normal Environment | Water 50 ml | Normal growth |
| T6 | Check | Water 50 ml | Flooded for 11 days |
| T7 | KHP-A 500x | Leaf spray 0.8 ml | after V2, then |
| T8 | KHP-B 500x | Leaf spray 0.8 ml | return to normal |
| T9 | KHP-A 100x | KHP infusion 50 ml | growth condition |

Fourteen days after V2 stage, the flooded groups (T6-T9) are returned to normal growth conditions, the inventors gather and measure the relevant indicators and take photos and noted the results as further stated herein.

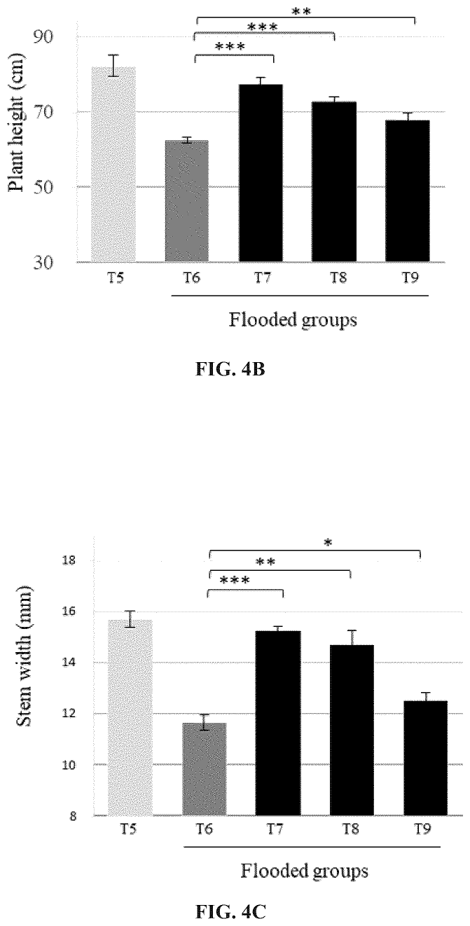

FIG. 4A shows the comparison of above-ground biomass dry weights among the 5 groups. T6, being the flooded group without any remedial assistance, has the lowest dry weight, naturally. The T7 and T8 groups dry weights even surpassed that of the unflooded T5 group.

For the plant heights, the T7, T8 and T9 groups all perform much better, either by leaf spray or by soil infusion, at V2 seedling stage, as shown in FIG. 4B.

FIG. 4C shows that the KHP solutions substantially boosted the stem width when applied at the V2 seedling stage.

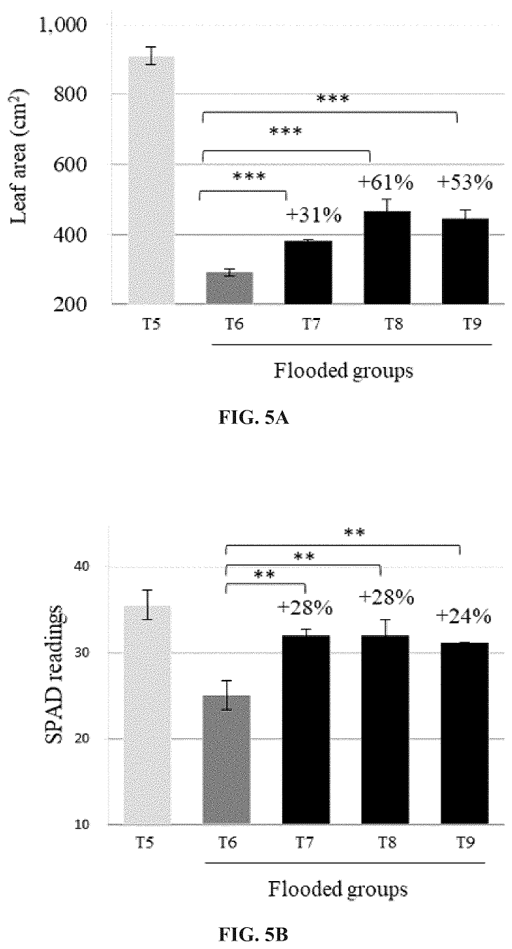

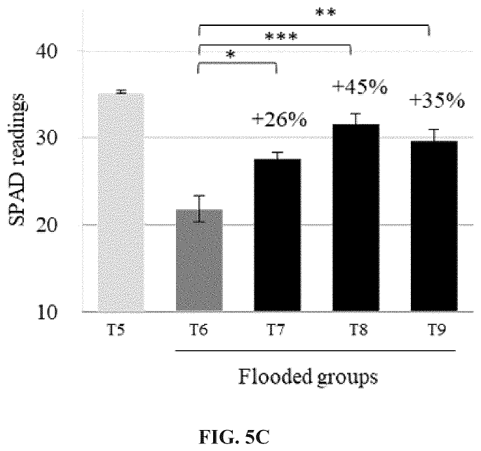

The remedial effect to the waterlogging stress, as reflected in the leaf development and chlorophyll count is shown in FIGS. 5A, 5B and 5C.

In FIG. 5A, the total leaf areas from the 5^{th} and 6^{th} leaves are tabulated. The increases in T7, T8 and T9 are 31%, 61% and 53% respectively over that of the T6.

FIG. 5B compares the SPAD reading on the 5^{th} leaf. The increases in T7, T8 and T9 are 28%, 28% and 24% respectively over that of the T6.

FIG. 5C compares the SPAD reading on the 6^{th} leaf. The increases in T7, T8 and T9 are 26%, 45% and 35% respectively over that of the T6.

As proven by the tests conducted and the scientific analysis/measurement done by the inventors, the method of creating the KHP solution and the method of application will help with the reduction of waterlogging stress inflicted upon corns due to flooding.

While the disclosure herein gave limited teachings and embodiment examples, it should be noted that the description and disclosure made herein illustrated the preferred embodiments of the invention and are not meant to limit the scope of the applicant's rights. Variations and alterations may be employed for yet additional embodiments without departing from the scope of the invention herein.

1. A method for reducing the suppression of corn growth due to waterlogging stress, comprising the steps of:
a. Preparing the KHP solution by mixing 50 kg of feathers whose content is 50% water and 40 kg of water in a sealed container;
b. hydrolyzing the mixture in the container with a temperature and pressure setting of 185° C. and 12 kg/cm² for a duration of 80 minutes;
c. using a mass spectrometer to confirm the combination of peptides in the solution to contain at least 253 peptides as listed in the specification where their molecular masses are between 593.3 and 3,508.9 Daltons and the concentration is in the range of 2.0×10⁵˜4.5×10⁵ ppm; and
d. infusing the solution to the soil at time of seeding the corns.

2. The method of claim 1 to reduce the suppression of corn growth wherein the solution is diluted with water by volume at the ratio of 1:50-500.

3. The method of claim 2 to reduce the suppression of corn growth wherein the application is done by leaf spraying the solution at seedling V2 stage.

4. A method for reducing the suppression of corn growth due to waterlogging stress, comprising the steps of:
a. putting 70 kg of feathers, whose water content is 46%, in a sealed container;
b. stirring the feathers in said sealed container;
c. hydrolyzing the feathers in the container with a temperature and pressure setting of 180° C. and 13 kg/cm² for a duration of 40 minutes;
d. using a mass spectrometer to confirm the combination of peptides in the solution to contain at least 253 peptides as listed in the specification whereby their molecular masses are between 705.9 and 3,194.7 Dalton and the concentration is in the range of 2.0×10⁵˜4.5×10⁵ ppm;
e. infusing the solution to the soil at time of seeding the corns.

5. The method of claim 4 to reduce the suppression of corn growth wherein the solution is diluted with water by volume at the ratio of 1:50-500.

6. The method of claim 5 to reduce the suppression of corn growth wherein the application is done by leaf spraying the solution at seedling V2 stage.


SEQUENCE LISTING (253 sequences)

SEQ ID NO: 1 - protein, 6 aa, Gallus gallus
FGLSGL

SEQ ID NO: 2 - protein, 7 aa, Gallus gallus
AAVGSSL

SEQ ID NO: 3 - protein, 7 aa, Gallus gallus
TAVGSST

SEQ ID NO: 4 - protein, 6 aa, Gallus gallus
LSLSSQ

SEQ ID NO: 5 - protein, 7 aa, Gallus gallus
FGISGLG

SEQ ID NO: 6 - protein, 6 aa, Gallus gallus
PILSSF

SEQ ID NO: 7 - protein, 7 aa, Gallus gallus
SLLSQGA

SEQ ID NO: 8 - protein, 6 aa, Gallus gallus
FNLRGA

SEQ ID NO: 9 - protein, 8 aa, Gallus gallus
SAAVGSSL

SEQ ID NO: 10 - protein, 6 aa, Gallus gallus
SRVVIQ

SEQ ID NO: 11 - protein, 6 aa, Gallus gallus
SRVVLE

SEQ ID NO: 12 - protein, 8 aa, Gallus gallus
GFGISGLG

SEQ ID NO: 13 - protein, 6 aa, Gallus gallus
SRFSGR

SEQ ID NO: 14 - protein, 6 aa, Gallus gallus
RVVIQP

SEQ ID NO: 15 - protein, 8 aa, Gallus gallus
SAAVGSIL

SEQ ID NO: 16 - protein, 7 aa, Gallus gallus
IVVIGHV

SEQ ID NO: 17 - protein, 9 aa, Gallus gallus
TAVGSSTSA

SEQ ID NO: 18 - protein, 7 aa, Gallus gallus
RVVIQPS

SEQ ID NO: 19 - protein, 7 aa, Gallus gallus
SRVVIQP

SEQ ID NO: 20 - protein, 7 aa, Gallus gallus
PQGEQLQ

SEQ ID NO: 21 - protein, 9 aa, Gallus gallus
RAGAGPPSP

SEQ ID NO: 22 - protein, 7 aa, Gallus gallus
DSRVVIQ

SEQ ID NO: 23 - protein, 8 aa, Gallus gallus
PGPILSSF

SEQ ID NO: 24 - protein, 9 aa, Gallus gallus
PISSGGFGI

SEQ ID NO: 25 - protein, 8 aa, Gallus gallus
ISGLGSRF

SEQ ID NO: 26 - protein, 10 aa, Gallus gallus
SGGFGISGLG

SEQ ID NO: 27 - protein, 7 aa, Gallus gallus
SRFSGRR

SEQ ID NO: 28 - protein, 9 aa, Gallus gallus
PGLALPVLP

SEQ ID NO: 29 - protein, 8 aa, Gallus gallus
SRVVIQPS

SEQ ID NO: 30 - protein, 8 aa, Gallus gallus
PILSSFPQ

SEQ ID NO: 31 - protein, 8 aa, Gallus gallus
LGSGIFWI

SEQ ID NO: 32 - protein, 9 aa, Gallus gallus
GISGLGSRF

SEQ ID NO: 33 - protein, 9 aa, Gallus gallus
VVVTLPGPI

SEQ ID NO: 34 - protein, 8 aa, Gallus gallus
CRLSLSQP

SEQ ID NO: 35 - protein, 11 aa, Gallus gallus
SAAVGSSLSAG

SEQ ID NO: 36 - protein, 11 aa, Gallus gallus
AVGSSTSAAVG

SEQ ID NO: 37 - protein, 9 aa, Gallus gallus
VVTLPGPIL

SEQ ID NO: 38 - protein, 9 aa, Gallus gallus
PGPILSSFP

SEQ ID NO: 39 - protein, 10 aa, Gallus gallus
SAAVGSILSE

SEQ ID NO: 40 - protein, 10 aa, Gallus gallus
VPISSGGFGI

SEQ ID NO: 41 - protein, 8 aa, Gallus gallus
QLAQRNTI

SEQ ID NO: 42 - protein, 9 aa, Gallus gallus
LSGLRSNLS

SEQ ID NO: 43 - protein, 10 aa, Gallus gallus
PSPVVVTLPG

SEQ ID NO: 44 - protein, 10 aa, Gallus gallus
PPPVVVTLPG

SEQ ID NO: 45 - protein, 10 aa, Gallus gallus
ISGLGSRFSG

SEQ ID NO: 46 - protein, 9 aa, Gallus gallus
LALRQSVEA

SEQ ID NO: 47 - protein, 10 aa, Gallus gallus
PLLGPPLLGI

SEQ ID NO: 48 - protein, 10 aa, Gallus gallus
VVTLPGPILS

SEQ ID NO: 49 - protein, 8 aa, Gallus gallus
VTLEDLRR

SEQ ID NO: 50 - protein, 9 aa, Gallus gallus
PILSSFPQN

SEQ ID NO: 51 - protein, 9 aa, Gallus gallus
PILSSFPQD

SEQ ID NO: 52 - protein, 11 aa, Gallus gallus
GTVGVSAGVTR

SEQ ID NO: 53 - protein, 9 aa, Gallus gallus
FGLSRVTLN

SEQ ID NO: 54 - protein, 12 aa, Gallus gallus
TAVGSSTSAAVG

SEQ ID NO: 55 - protein, 10 aa, Gallus gallus
VVVTLPGPIL

SEQ ID NO: 56 - protein, 10 aa, Gallus gallus
PPVVVTFPGP

SEQ ID NO: 57 - protein, 10 aa, Gallus gallus
PPPVVVTFPG

SEQ ID NO: 58 - protein, 10 aa, Gallus gallus
VVIQPSPVVV

SEQ ID NO: 59 - protein, 10 aa, Gallus gallus
FGISGLGSRF

SEQ ID NO: 60 - protein, 10 aa, Gallus gallus
PGPILSSFPQ

SEQ ID NO: 61 - protein, 10 aa, Gallus gallus
FGWSGLGRGL

SEQ ID NO: 62 - protein, 9 aa, Gallus gallus
LEAQLSELR

SEQ ID NO: 63 - protein, 9 aa, Gallus gallus
IVGAWYTVR

SEQ ID NO: 64 - protein, 11 aa, Gallus gallus
LLSPPGLPGLL

SEQ ID NO: 65 - protein, 11 aa, Gallus gallus
VVVTLPGPILS

SEQ ID NO: 66 - protein, 11 aa, Gallus gallus
GFGISGLGSRF

SEQ ID NO: 67 - protein, 11 aa, Gallus gallus
PVVVTLPGPIL

SEQ ID NO: 68 - protein, 11 aa, Gallus gallus
VSQGEITVPSI

SEQ ID NO: 69 - protein, 11 aa, Gallus gallus
SILSEEGVPIS

SEQ ID NO: 70 - protein, 11 aa, Gallus gallus
PVVVTFPGPIL

SEQ ID NO: 71 - protein, 13 aa, Gallus gallus
PISSGGFGISGLG

SEQ ID NO: 72 - protein, 13 aa, Gallus gallus
VGSSTSAAVGSIL

SEQ ID NO: 73 - protein, 11 aa, Gallus gallus
GFLSSAVSLNR

SEQ ID NO: 74 - protein, 12 aa, Gallus gallus
GGFGISGLGSRF

SEQ ID NO: 75 - protein, 11 aa, Gallus gallus
PGPILSSFPQN

SEQ ID NO: 76 - protein, 11 aa, Gallus gallus
PGPILSSFPQD

SEQ ID NO: 77 - protein, 12 aa, Gallus gallus
GGFGWSGLGRGL

SEQ ID NO: 78 - protein, 12 aa, Gallus gallus
PSPVVVTLPGPI

SEQ ID NO: 79 - protein, 12 aa, Gallus gallus
VVVTLPGPILSS

SEQ ID NO: 80 - protein, 12 aa, Gallus gallus
PVVVTLPGPILS

SEQ ID NO: 81 - protein, 12 aa, Gallus gallus
SPVVVTLPGPIL

SEQ ID NO: 82 - protein, 11 aa, Gallus gallus
RVVIQPSPVVV

SEQ ID NO: 83 - protein, 11 aa, Gallus gallus
QALTEVYAKAN

SEQ ID NO: 84 - protein, 10 aa, Gallus gallus
IETLTQFLGW

SEQ ID NO: 85 - protein, 13 aa, Gallus gallus
GFGSRSLVGLGGT

SEQ ID NO: 86 - protein, 14 aa, Gallus gallus
AVGSSTSAAVGSIL

SEQ ID NO: 87 - protein, 12 aa, Gallus gallus
PVVVTFPGPILS

SEQ ID NO: 88 - protein, 14 aa, Gallus gallus
VGSSTSAAVGSILS

SEQ ID NO: 89 - protein, 13 aa, Gallus gallus
GFGISGLGSRFSG

SEQ ID NO: 90 - protein, 13 aa, Gallus gallus
SGGFGISGLGSRF

SEQ ID NO: 91 - protein, 14 aa, Gallus gallus
VPISSGGFGISGLG

SEQ ID NO: 92 - protein, 12 aa, Gallus gallus
VVIQPSPVVVTL

SEQ ID NO: 93 - protein, 13 aa, Gallus gallus
SILSEEGVPISSG

SEQ ID NO: 94 - protein, 13 aa, Gallus gallus
VMGCKAAGASRII

SEQ ID NO: 95 - protein, 13 aa, Gallus gallus
SPVVVTLPGPILS

SEQ ID NO: 96 - protein, 12 aa, Gallus gallus
SRVVIQPSPVVV

SEQ ID NO: 97 - protein, 13 aa, Gallus gallus
PSPVVVTLPGPIL

SEQ ID NO: 98 - protein, 12 aa, Gallus gallus
RVVIQPSPVVVT

SEQ ID NO: 99 - protein, 13 aa, Gallus gallus
PPPVVVTLPGPIL

SEQ ID NO: 100 - protein, 15 aa, Gallus gallus
GVPISSGGFGISGLG

SEQ ID NO: 101 - protein, 13 aa, Gallus gallus
VIQPSPVVVTLPG

SEQ ID NO: 102 - protein, 15 aa, Gallus gallus
AVGSSTSAAVGSILS

SEQ ID NO: 103 - protein, 15 aa, Gallus gallus
TAVGSSTSAAVGSIL

SEQ ID NO: 104 - protein, 14 aa, Gallus gallus
SGGFGISGLGSRFS

SEQ ID NO: 105 - protein, 13 aa, Gallus gallus
VVVTLPGPILSSF

SEQ ID NO: 106 - protein, 13 aa, Gallus gallus
PPPVVVTFPGPIL

SEQ ID NO: 107 - protein, 14 aa, Gallus gallus
GLGGFGWSGLGRGL

SEQ ID NO: 108 - protein, 13 aa, Gallus gallus
VTLPGPILSSFPQ

SEQ ID NO: 109 - protein, 14 aa, Gallus gallus
AVGSILSEEGVPIS

SEQ ID NO: 110 - protein, 14 aa, Gallus gallus
SLGLGGFGWSGLGR

SEQ ID NO: 111 - protein, 14 aa, Gallus gallus
PSPVVVTLPGPILS

SEQ ID NO: 112 - protein, 13 aa, Gallus gallus
SRVVIQPSPVVVT

SEQ ID NO: 113 - protein, 15 aa, Gallus gallus
SGGFGISGLGSRFSG

SEQ ID NO: 114 - protein, 14 aa, Gallus gallus
PPPVVVTLPGPILS

SEQ ID NO: 115 - protein, 14 aa, Gallus gallus
VAIQPPPVVVTLPG

SEQ ID NO: 116 - protein, 15 aa, Gallus gallus
PISSGGFGISGLGSR

SEQ ID NO: 117 - protein, 13 aa, Gallus gallus
PIVDVAEALLHVK

SEQ ID NO: 118 - protein, 14 aa, Gallus gallus
VVIQPSPVVVTLPG

SEQ ID NO: 119 - protein, 13 aa, Gallus gallus
RVVIQPSPVVVTL

SEQ ID NO: 120 - protein, 16 aa, Gallus gallus
TAVGSSTSAAVGSILS

SEQ ID NO: 121 - protein, 14 aa, Gallus gallus
PPPVVVTFPGPILS

SEQ ID NO: 122 - protein, 15 aa, Gallus gallus
SLGLGGFGWSGLGRG

SEQ ID NO: 123 - protein, 14 aa, Gallus gallus
VVVTLPGPILSSFP

SEQ ID NO: 124 - protein, 14 aa, Gallus gallus
PVVVTLPGPILSSF

SEQ ID NO: 125 - protein, 15 aa, Gallus gallus
AAVGSILSEEGVPIS

SEQ ID NO: 126 - protein, 15 aa, Gallus gallus
SAAVGSILSEEGVPI

SEQ ID NO: 127 - protein, 16 aa, Gallus gallus
EGVPISSGGFGISGLG

SEQ ID NO: 128 - protein, 14 aa, Gallus gallus
VVTLPGPILSSFPQ

SEQ ID NO: 129 - protein, 15 aa, Gallus gallus
PSPVVVTLPGPILSS

SEQ ID NO: 130 - protein, 16 aa, Gallus gallus
GKDGPKGVRGDAGPPG

SEQ ID NO: 131 - protein, 15 aa, Gallus gallus
SVAIQPPPVVVTLPG

SEQ ID NO: 132 - protein, 14 aa, Gallus gallus
SRVVIQPSPVVVTL

SEQ ID NO: 133 - protein, 14 aa, Gallus gallus
LHTEAAATVPDRSE

SEQ ID NO: 134 - protein, 15 aa, Gallus gallus
VVIQPSPVVVTLPGP

SEQ ID NO: 135 - protein, 14 aa, Gallus gallus
RVVIQPSPVVVTLP

SEQ ID NO: 136 - protein, 16 aa, Gallus gallus
SAAVGSILSEEGVPIS

SEQ ID NO: 137 - protein, 15 aa, Gallus gallus
PVVVTLPGPILSSFP

SEQ ID NO: 138 - protein, 15 aa, Gallus gallus
IQPSPVVVTLPGPIL

SEQ ID NO: 139 - protein, 17 aa, Gallus gallus
SVPIGAGGSLGLGGFGW

SEQ ID NO: 140 - protein, 16 aa, Gallus gallus
SLGLGGFGWSGLGRGL

SEQ ID NO: 141 - protein, 17 aa, Gallus gallus
FGGGFGGGDGILPAGEK

SEQ ID NO: 142 - protein, 17 aa, Gallus gallus
TAVGSSTSAAVGSILSE

SEQ ID NO: 143 - protein, 16 aa, Gallus gallus
PISSGGFGISGLGSRF

SEQ ID NO: 144 - protein, 19 aa, Gallus gallus
SAAVGSSLSAGSVPIGAGG

SEQ ID NO: 145 - protein, 17 aa, Gallus gallus
GVPISSGGFGISGLGSR

SEQ ID NO: 146 - protein, 15 aa, Gallus gallus
VVVTLPGPILSSFPQ

SEQ ID NO: 147 - protein, 15 aa, Gallus gallus
RVVIQPSPVVVTLPG

SEQ ID NO: 148 - protein, 15 aa, Gallus gallus
SRVVIQPSPVVVTLP

SEQ ID NO: 149 - protein, 16 aa, Gallus gallus
PSPVVVTLPGPILSSF

SEQ ID NO: 150 - protein, 16 aa, Gallus gallus
AIQPPPVVVTLPGPIL

SEQ ID NO: 151 - protein, 16 aa, Gallus gallus
IQPSPVVVTLPGPILS

SEQ ID NO: 152 - protein, 16 aa, Gallus gallus
VIQPSPVVVTLPGPIL

SEQ ID NO: 153 - protein, 17 aa, Gallus gallus
VPISSGGFGISGLGSRF

SEQ ID NO: 154 - protein, 16 aa, Gallus gallus
SRVVIQPSPVVVTLPG

SEQ ID NO: 155 - protein, 17 aa, Gallus gallus
SILSEEGVPISSGGFGI

SEQ ID NO: 156 - protein, 16 aa, Gallus gallus
PVVVTLPGPILSSFPQ

SEQ ID NO: 157 - protein, 16 aa, Gallus gallus
RVVIQPSPVVVTLPGP

SEQ ID NO: 158 - protein, 18 aa, Gallus gallus
SAAVGSILSEEGVPISSG

SEQ ID NO: 159 - protein, 16 aa, Gallus gallus
VVVTLPGPILSSFPQN

SEQ ID NO: 160 - protein, 18 aa, Gallus gallus
GVPISSGGFGISGLGSRF

SEQ ID NO: 161 - protein, 17 aa, Gallus gallus
PSPVVVTLPGPILSSFP

SEQ ID NO: 162 - protein, 17 aa, Gallus gallus
VAIQPPPVVVTLPGPIL

SEQ ID NO: 163 - protein, 17 aa, Gallus gallus
VIQPSPVVVTLPGPILS

SEQ ID NO: 164 - protein, 17 aa, Gallus gallus
VVIQPSPVVVTLPGPIL

SEQ ID NO: 165 - protein, 17 aa, Gallus gallus
SPVVVTLPGPILSSFPQ

SEQ ID NO: 166 - protein, 15 aa, Gallus gallus
VILSNTKTQYSIKIH

SEQ ID NO: 167 - protein, 17 aa, Gallus gallus
SRVVIQPSPVVVTLPGP

SEQ ID NO: 168 - protein, 17 aa, Gallus gallus
DSRVVIQPSPVVVTLPG

SEQ ID NO: 169 - protein, 17 aa, Gallus gallus
PVVVTLPGPILSSFPQN

SEQ ID NO: 170 - protein, 17 aa, Gallus gallus
RVVIQPSPVVVTLPGPI

SEQ ID NO: 171 - protein, 19 aa, Gallus gallus
GVPISSGGFGISGLGSRFS

SEQ ID NO: 172 - protein, 18 aa, Gallus gallus
SVAIQPPPVVVTLPGPIL

SEQ ID NO: 173 - protein, 18 aa, Gallus gallus
VAIQPPPVVVTLPGPILS

SEQ ID NO: 174 - protein, 18 aa, Gallus gallus
VVIQPSPVVVTLPGPILS

SEQ ID NO: 175 - protein, 18 aa, Gallus gallus
PSPVVVTLPGPILSSFPQ

SEQ ID NO: 176 - protein, 20 aa, Gallus gallus
GVPISSGGFGISGLGSRFSG

SEQ ID NO: 177 - protein, 18 aa, Gallus gallus
AVPLVRPVTMVPSIPGIP

SEQ ID NO: 178 - protein, 18 aa, Gallus gallus
PPPVVVTLPGPILSSFPQ

SEQ ID NO: 179 - protein, 18 aa, Gallus gallus
SPVVVTLPGPILSSFPQN

SEQ ID NO: 180 - protein, 18 aa, Gallus gallus
LPGPGLQLLHGTTTLAFK

SEQ ID NO: 181 - protein, 18 aa, Gallus gallus
PPPVVVTFPGPILSSFPQ

SEQ ID NO: 182 - protein, 19 aa, Gallus gallus
SVAIQPPPVVVTLPGPILS

SEQ ID NO: 183 - protein, 19 aa, Gallus gallus
SSVAIQPPPVVVTLPGPIL

SEQ ID NO: 184 - protein, 18 aa, Gallus gallus
RVVIQPSPVVVTLPGPIL

SEQ ID NO: 185 - protein, 19 aa, Gallus gallus
PSPVVVTLPGPILSSFPQN

SEQ ID NO: 186 - protein, 21 aa, Gallus gallus
SILSEEGVPISSGGFGISGLG

SEQ ID NO: 187 - protein, 19 aa, Gallus gallus
SRVVIQPSPVVVTLPGPIL

SEQ ID NO: 188 - protein, 19 aa, Gallus gallus
RVVIQPSPVVVTLPGPILS

SEQ ID NO: 189 - protein, 22 aa, Gallus gallus
SVPIGAGGSLGLGGFGWSGLGR

SEQ ID NO: 190 - protein, 22 aa, Gallus gallus
TAVGSSTSAAVGSILSEEGVPI

SEQ ID NO: 191 - protein, 22 aa, Gallus gallus
SAAVGSILSEEGVPISSGGFGI

SEQ ID NO: 192 - protein, 21 aa, Gallus gallus
SEEGVPISSGGFGISGLGSRF

SEQ ID NO: 193 - protein, 20 aa, Gallus gallus
SRVVIQPSPVVVTLPGPILS

SEQ ID NO: 194 - protein, 23 aa, Gallus gallus
SVPIGAGGSLGLGGFGWSGLGRG

SEQ ID NO: 195 - protein, 20 aa, Gallus gallus
DSRVVIQPSPVVVTLPGPIL

SEQ ID NO: 196 - protein, 23 aa, Gallus gallus
TAVGSSTSAAVGSILSEEGVPIS

SEQ ID NO: 197 - protein, 22 aa, Gallus gallus
LRGDVGPVGRTGEQGIAGPPGF

SEQ ID NO: 198 - protein, 21 aa, Gallus gallus
SRVVIQPSPVVVTLPGPILSS

SEQ ID NO: 199 - protein, 21 aa, Gallus gallus
VVIQPSPVVVTLPGPILSSFP

SEQ ID NO: 200 - protein, 22 aa, Gallus gallus
GVPISSGGFGISGLGSRFSGRR

SEQ ID NO: 201 - protein, 24 aa, Gallus gallus
SVPIGAGGSLGLGGFGWSGLGRGL

SEQ ID NO: 202 - protein, 21 aa, Gallus gallus
DSRVVIQPSPVVVTLPGPILS

SEQ ID NO: 203 - protein, 21 aa, Gallus gallus
VIQPSPVVVTLPGPILSSFPQ

SEQ ID NO: 204 - protein, 24 aa, Gallus gallus
AVGSILSEEGVPISSGGFGISGLG

SEQ ID NO: 205 - protein, 21 aa, Gallus gallus
RVVIQPSPVVVTLPGPILSSF

SEQ ID NO: 206 - protein, 23 aa, Gallus gallus
SILSEEGVPISSGGFGISGLGSR

SEQ ID NO: 207 - protein, 21 aa, Gallus gallus
IPSSVPESSFKIKGNSSSFHI

SEQ ID NO: 208 - protein, 22 aa, Gallus gallus
VAIQPPPVVVTLPGPILSSFPQ

SEQ ID NO: 209 - protein, 25 aa, Gallus gallus
TAVGSSTSAAVGSILSEEGVPISSG

SEQ ID NO: 210 - protein, 22 aa, Gallus gallus
VVIQPSPVVVTLPGPILSSFPQ

SEQ ID NO: 211 - protein, 23 aa, Gallus gallus
PSPVVVTLPGPILSSFPQNTAVG

SEQ ID NO: 212 - protein, 20 aa, Gallus gallus
KENFEVLCKDGTRKPVTDAE

SEQ ID NO: 213 - protein, 21 aa, Gallus gallus
SIQACKLAQSHGWGVMVSHRS

SEQ ID NO: 214 - protein, 22 aa, Gallus gallus
SRVVIQPSPVVVTLPGPILSSF

SEQ ID NO: 215 - protein, 22 aa, Gallus gallus
RVVIQPSPVVVTLPGPILSSFP

SEQ ID NO: 216 - protein, 23 aa, Gallus gallus
EQPPLCVASVSVKEPASETPAVP

SEQ ID NO: 217 - protein, 23 aa, Gallus gallus
SVAIQPPPVVVTLPGPILSSFPQ

SEQ ID NO: 218 - protein, 26 aa, Gallus gallus
SAAVGSILSEEGVPISSGGFGISGLG

SEQ ID NO: 219 - protein, 24 aa, Gallus gallus
SILSEEGVPISSGGFGISGLGSRF

SEQ ID NO: 220 - protein, 24 aa, Gallus gallus
PSPVVVTLPGPILSSFPQNTAVGS

SEQ ID NO: 221 - protein, 23 aa, Gallus gallus
VVIQPSPVVVTLPGPILSSFPQN

SEQ ID NO: 222 - protein, 23 aa, Gallus gallus
SRVVIQPSPVVVTLPGPILSSFP

SEQ ID NO: 223 - protein, 24 aa, Gallus gallus
SSVAIQPPPVVVTLPGPILSSFPQ

SEQ ID NO: 224 - protein, 23 aa, Gallus gallus
RVVIQPSPVVVTLPGPILSSFPQ

SEQ ID NO: 225 - protein, 25 aa, Gallus gallus
SILSEEGVPISSGGFGISGLGSRFS

SEQ ID NO: 226 - protein, 27 aa, Gallus gallus
TSAAVGSILSEEGVPISSGGFGISGLG

SEQ ID NO: 227 - protein, 26 aa, Gallus gallus
SILSEEGVPISSGGFGISGLGSRFSG

SEQ ID NO: 228 - protein, 24 aa, Gallus gallus
SRVVIQPSPVVVTLPGPILSSFPQ

SEQ ID NO: 229 - protein, 28 aa, Gallus gallus
TAVGSSTSAAVGSILSEEGVPISSGGFG

SEQ ID NO: 230 - protein, 28 aa, Gallus gallus
STSAAVGSILSEEGVPISSGGFGISGLG

SEQ ID NO: 231 - protein, 24 aa, Gallus gallus
RVVIQPSPVVVTLPGPILSSFPQN

SEQ ID NO: 232 - protein, 25 aa, Gallus gallus
SRVVIQPSPVVVTLPGPILSSFPQN

SEQ ID NO: 233 - protein, 25 aa, Gallus gallus
DSRVVIQPSPVVVTLPGPILSSFPQ

SEQ ID NO: 234 - protein, 29 aa, Gallus gallus
TAVGSSTSAAVGSILSEEGVPISSGGFGI

SEQ ID NO: 235 - protein, 29 aa, Gallus gallus
SAAVGSILSEEGVPISSGGFGISGLGSRF

SEQ ID NO: 236 - protein, 24 aa, Gallus gallus
VEQLVNLQHLDVAYNLLLEHAQLA

SEQ ID NO: 237 - protein, 28 aa, Gallus gallus
SVAIQPPPVVVTLPGPILSSFPQSTAVG

SEQ ID NO: 238 - protein, 28 aa, Gallus gallus
SILSEEGVPISSGGFGISGLGSRFSGRR

SEQ ID NO: 239 - protein, 30 aa, Gallus gallus
TSAAVGSILSEEGVPISSGGFGISGLGSRF

SEQ ID NO: 240 - protein, 29 aa, Gallus gallus
SSVAIQPPPVVVTLPGPILSSFPQSTAVG

SEQ ID NO: 241 - protein, 31 aa, Gallus gallus
STSAAVGSILSEEGVPISSGGFGISGLGSRF

SEQ ID NO: 242 - protein, 31 aa, Gallus gallus
PSPVVVTLPGPILSSFPQNTAVGSSTSAAVG

SEQ ID NO: 243 - protein, 33 aa, Gallus gallus
TAVGSSTSAAVGSILSEEGVPISSGGFGISGLG

SEQ ID NO: 244 - protein, 31 aa, Gallus gallus
AVGSILSEEGVPISSGGFGISGLGSRFSGRR

SEQ ID NO: 245 - protein, 27 aa, Gallus gallus
NKVLVHCAMGRSRSATLVLAYLMIYKN

SEQ ID NO: 246 - protein, 32 aa, Gallus gallus
SSVAIQPPPVVVTLPGPILSSFPQSTAVGSTA

SEQ ID NO: 247 - protein, 32 aa, Gallus gallus
AAVGSILSEEGVPISSGGFGISGLGSRFSGRR

SEQ ID NO: 248 - protein, 35 aa, Gallus gallus
TAVGSSTSAAVGSILSEEGVPISSGGFGISGLGSR

SEQ ID NO: 249 - protein, 33 aa, Gallus gallus
SAAVGSILSEEGVPISSGGFGISGLGSRFSGRR

SEQ ID NO: 250 - protein, 34 aa, Gallus gallus
PSPVVVTLPGPILSSFPQNTAVGSSTSAAVGSIL

SEQ ID NO: 251 - protein, 35 aa, Gallus gallus
PSPVVVTLPGPILSSFPQNTAVGSSTSAAVGSILS

SEQ ID NO: 252 - protein, 36 aa, Gallus gallus
TAVGSSTSAAVGSILSEEGVPISSGGFGISGLGSRF

SEQ ID NO: 253 - protein, 29 aa, Gallus gallus
RPRVYLHCTNPRHPKYREGDVTLFALNLS